(19) United States
(12) Patent Application Publication
(10) Pub. No.: US 2018/0299419 A1
(43) Pub. Date: Oct. 18, 2018

(54) INTERNET OF VEHICLES-BASED AIR QUALITY DETECTING SYSTEM

(71) Applicants: ZHEJIANG GEELY HOLDING GROUP CO., LTD, Hangzhou (CN); ZHEJIANG GEELY AUTOMOBILE RESEARCH INSTITUTE CO., LTD, Taizhou (CN)

(72) Inventor: Shufu LI, Hangzhou (CN)

(21) Appl. No.: 15/759,094

(22) PCT Filed: Sep. 20, 2016
(86) PCT No.: PCT/CN2016/099463

§ 371 Date: Mar. 9, 2018

(30) Foreign Application Priority Data
Sep. 24, 2015 (CN) 201510617066.X

(51) Int. Cl. G01N 33/00; B60H 1/00; G07C 5/00; H04W 4/40

(52) U.S. Cl. CPC G01N 33/0075; B60H 1/008; H04W 4/40; G07C 5/008; G01N 33/0031

(57) ABSTRACT

An internet of vehicles-based air quality detecting system, comprises an air quality detecting device arranged in each vehicle of a plurality of vehicles, the air quality detecting device comprising: an air quality detecting unit, used for acquiring air quality data of the outer part of the vehicle in a current position where the corresponding vehicle is located, and a data sending unit, used for sending the air quality data to a remote server. The internet of vehicles-based air quality detecting system uses each vehicle accessed to an internet of vehicles as a movable air quality monitoring point, thereby acquiring air quality data of the outer parts of the vehicles in current positions where all vehicles are located; and there may be a large quantity of the vehicles accessed to the internet of vehicles, thereby being able to acquire accurate air quality data, and making air quality detection more accurate.

### TECHNICAL FIELD

The present invention relates to the field of air quality detection, and in particular to an air quality detection system based on the Internet of Vehicles.

### BACKGROUND OF THE INVENTION

Fog and haze weather is a state of atmospheric pollution, and the expression of fog and haze is a general expression for the content of various suspended particulate matters in the haze exceeding the standards. In short, the fine drops of water in air is fog, which belongs to a liquid; the haze is a solid, is composed of tiny dust particles, sulfuric acid, nitric acid, inhalable particulate matter and other particles in air, and can make the air become turbid to reduce the visibility; and the fog and the haze are combined to form fog and haze.

Now most of the domestic large and medium-sized cities are facing the invasion of haze, whereby causing great trouble to the people's production and life, so that the prevention and control of fog and haze become national key issues to be addressed, in which the fog and haze broadcast plays an active guiding role on the prevention and control of fog and haze. At present, the air pollution index used in the fog and haze broadcast is obtained directly from the air quality detection data of fixed monitoring points, or the collected fixed point detection data are calculated by a mathematical model and converted into the overall air pollution index of the local city, which is thereby used to guide people's production and life.

However, people often feel that the air pollution index in the fog and haze broadcast is deviated from their actual feeling so as to suspect the accuracy of the air quality data.

### SUMMARY OF THE INVENTION

An object of the present invention is to, for the purpose of performing air quality detection more accurately to obtain accurate air quality data, provide an air quality detection system based on the Internet of Vehicles.

The inventor has found that there are a number of reasons why people feel that the air quality data are inaccurate, and one of the key reasons is that the distribution of air quality monitoring points is too sparse. For example, it is reported in a newspaper in Beijing in 2015 that there are 35 air quality monitoring points in Beijing, only a few of which being distributed in major urban areas, only a few air quality monitoring points are provided in medium-sized city, and even no air quality monitoring point is provided in small cities. The inventor has also found that, if the air quality monitoring points are substantially increased in the existing manner, the air detection cost will increase significantly, especially when the number of air quality monitoring points is increased to several orders of magnitude of the number of the existing air quality monitoring points, the cost will increase dramatically. Also, the determination and selection of the layouts to be considered for arranging a large number of air quality monitoring points is also a challenge. Therefore, the present invention provides an air quality detection system based on the Internet of Vehicles.

In particular, the present invention provides an internet of vehicles-based air quality detecting system, comprising an air quality detection device arranged in each of a plurality of vehicles, the air quality detection device comprising:

an air quality detection unit for acquiring air quality data outside the vehicle at the current position where the corresponding vehicle is located; and

a data transmission unit for transmitting the air quality data to a remote server.

Further, the air quality detection device also comprises a position acquisition unit for acquiring position data of the current position where the corresponding vehicle is located,

wherein the data transmission unit is configured to transmit the position data of the current position to the server together with the air quality data of the current position.

Further, the air quality detection device also comprises a time acquisition unit for acquiring time data of a moment when the corresponding vehicle is located at the current position,

wherein the data transmission unit is configured to transmit the time data of the current position to the server together with the air quality data of the current position.

Further, the air quality detection unit is configured to acquire the air quality data corresponding to a schedule, which comprises one or more discrete scheduled time; and

the data transmission unit is configured to transmit only the air quality data corresponding to the schedule to the server.

Further, the plurality of air quality detection units corresponding to the plurality of vehicles use the same schedule.

Further, the system also comprises the server, which is configured to form an air quality map according to the air quality data from the plurality of vehicles.

Further, the air quality detection unit has an inlet port, which is in communication with an outer air inlet pipe and an inner air inlet pipe separately through a one-way valve, an air inlet of the outer air inlet pipe is provided outside the vehicle, and an air inlet of the inner air inlet pipe is provided inside the vehicle, and by selecting the opening direction of the one-way valve, the inlet port is in communication with one of the outer air inlet pipe and the inner air inlet pipe so as to correspondingly acquire the air quality data outside the vehicle or acquire the in-vehicle air quality data inside the vehicle.

Further, the air inlet of the outer air inlet pipe is provided at an intake grille of the vehicle or in a transition region between a windshield and an engine hood of the vehicle.

Further, the system also comprises:

an air circulation determination unit for determining whether or not a door or a window of the vehicle has been in an open state for a predetermined time,

wherein the air quality detection unit is installed inside the vehicle and acquires in-vehicle air quality data inside the vehicle; and the data transmission unit is configured to, if the result of the determination of the air circulation determination unit is positive, transmit the in-vehicle air quality data as the air quality data outside the vehicle to the server.

Further, the air quality data is or includes PM2.5 data.

The air quality detection system based on the Internet of Vehicles of the present invention uses each of the vehicles accessed to the Internet of Vehicles as an active air quality monitoring point so as to obtain the air quality data outside the vehicle at the current position where each vehicle is located, and can transmit the air quality data to a server as a data processing center through the Internet of Vehicles in time. Since there may be a very large number of vehicles accessed to the Internet of Vehicles, the present invention can obtain air quality data of many orders of magnitude more than those from the existing fixed monitoring points, so that the sample size sampled by the present invention is relatively comprehensive, and thus the present invention can obtain accurate air quality data, and the air quality detection using the system of the present invention is more accurate.

According to the detailed description of the particular embodiments of the present invention below in conjunction with the accompanying drawings, the above-mentioned and other objects, advantages and features of the present invention will be much clearer to a person skilled in the art.

### BRIEF DESCRIPTION OF THE DRAWINGS

Some of the particular embodiments of the present invention will be described below in detail in an exemplary but not limiting way with reference to the accompanying drawings. The same reference signs in the figures indicate the same or similar components or parts. A person skilled in the art should understand that these figures are not necessarily drawn to scale. In the accompanying drawings:

FIG. 1 is a schematic logic control diagram of an air quality detection system based on the Internet of Vehicles according to one embodiment of the present invention;

FIG. 2 is a schematic structural diagram of the air quality detection device shown in FIG. 1;

FIG. 3 is a schematic assembly diagram of the air quality monitoring unit shown in FIG. 2 with an outer air inlet pipe and an inner air inlet pipe;

FIG. 4 is another schematic logic control diagram of the air quality detection device shown in FIG. 1.

The symbols in the drawings represent the following meanings:
1 Vehicle, 2 Outer air inlet pipe, 3 Inner air inlet pipe, 4 One-way valve,
100 Air quality detection device,
10 Air quality detection unit,
11 Inlet port,
20 Data transmission unit,
30 Position acquisition unit,
40 Time acquisition unit,
200 Server,
300 Air circulation determination unit.

### DETAILED DESCRIPTION OF THE INVENTION

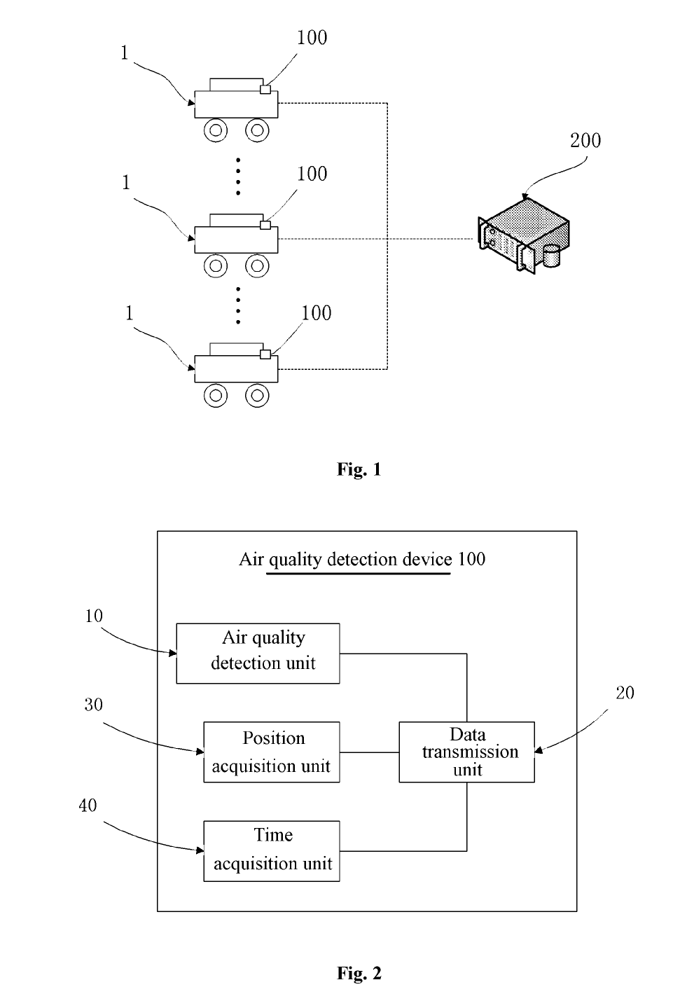

FIG. 1 is a schematic structural diagram of an air quality detection system based on the Internet of Vehicles according to one embodiment of the present invention. In FIG. 1, each vehicle 1 and the server 200 may communicate with each other through the Internet of Vehicles. As shown in FIG. 1, the air quality detection system may comprise an air quality detection device 100 arranged at each vehicle 1 of a plurality of vehicles 1.

In this embodiment, the current position of the vehicle 1 may be any position on the traveling route of the vehicle 1, and may also be a position where the vehicle 1 is parked. In general, the area where the vehicle arrives at is also often an area where the human activities are frequent, so that the air quality data that can be obtained from the positions on the traveling route of the vehicle 1 is more representative of the air quality of the people's actual living area, so that the air quality situations in the fog and haze broadcast are more consistent with the people's actual feelings.

As the annual sale of a large-scale automobile enterprise is generally more than 300 thousand, up to more than 500 thousand, and the vehicles are sold to the provinces, cities and counties all over the country, the air quality detection system based on the Internet of Vehicles of the present invention, therefore, covers the monitoring points much more than the fixed monitoring points arranged by the country, so the detection range can cover most of the country.

For the air quality detection system based on the Internet of Vehicles of the present invention, by arranging an air quality detection device 100 at each vehicle 1 of the plurality of vehicle 1 and by obtaining the air quality data outside the vehicle 1 at the current position where the corresponding vehicle 1 is located by means of the air quality detection device 100, the present invention, therefore, can obtain the air quality data of many orders of magnitude more than those from the existing fixed monitoring points, so that the sample size sampled by the present invention is relatively comprehensive, and thus the present invention can obtain accurate air quality data, so that the air quality detection of the system of the present invention is more accurate and closer to the actual situation, so as to provide more accurate guidance for the people's travel and life.

FIG. 2 is a schematic logic control diagram of the air quality detection device 100 shown in FIG. 1. With reference to FIG. 2, the air quality detection device 100 of each vehicle 1 may comprise an air quality detection unit 10 and a data transmission unit 20. The air quality detection unit 10 is used for acquiring air quality data outside the vehicle 1 at the current position where the corresponding vehicle 1 is located. The data transmission unit 20 is used for transmitting the air quality data to a remote server 200. Specifically, in this embodiment, the data transmission unit 20 may be a vehicle-mounted data transmission system or a part thereof by which the vehicle 1 is accessed to the Internet of Vehicles, so as to take full advantage of the original configuration of the vehicle 1 and reduce the cost. Of course, the data transmission unit 20 may also be an additionally configured unit.

With reference to FIG. 2, in this embodiment, the air quality detection device 100 may further comprise a position acquisition unit 30 for acquiring position data of the current position where the corresponding vehicle 1 is located. The data transmission unit 20 may be configured to transmit the position data of the current position to the server 200 (see FIG. 1) together with the air quality data of the current position. In this way, by simultaneously acquiring the air quality data and the position data of the current position where the vehicle 1 is located and transmitting the data together to the server 200 (see FIG. 1), it is possible to facilitate the drawing of an air quality map on the side of server 200 (see FIG. 1), thus forming a healthy track of life and areas to better guide people's work and travel.

In this embodiment, the position acquisition unit 30 may be a GPS system built in the vehicle 1, so as to take full advantage of the original configuration of the vehicle 1 and reduce the cost. Of course, in other embodiments, the position acquisition unit 30 may also be an additionally provided unit.

It should be understood that, with reference to FIG. 1, when the air quality map is drawn on the side of server 200, it may be necessary to take into consideration of the problem of matching or alignment in time of the air quality data from the vehicles 1. For example, the data from a vehicle 1 may be delayed to reach the server 200 due to network congestion, so that the data received by the server 200 at a certain time may reflect the air quality data in different times.

To this end, with reference to FIG. 2, in this embodiment, the air quality detection device 100 may further comprise a time acquisition unit 40 for acquiring time data of a moment when the corresponding vehicle 1 is located at the current position. The data transmission unit 20 may be configured to transmit the time data of the current position to the server 200 (see FIG. 1) together with the air quality data of the current position. In another embodiment, the data transmission unit 20 may further be configured to transmit the time data of the current position, the air quality data of the current position and the position data of the current position together to the server 200 (see FIG. 1). In this way, on the one hand, it is possible to screen out the obsolete air quality data according to the time data, and on the other hand, when air quality maps are drawn, the air quality maps from the respective vehicles at the same time or in the same period of time (i.e., the data aligned in time) may be used to draw the air quality maps corresponding to this time or this period of time, thereby enabling the air quality map to be time-dependent, and it is possible to observe the change of the air quality maps over time according to the data at different times.

The air quality detection unit 10 may be configured to acquire the air quality data corresponding to a schedule, which comprises one or more discrete scheduled time. The data transmission unit 20 may be configured to transmit only the air quality data corresponding to the schedule to the server 200. A scheduled time in this embodiment refers to any time of the predetermined day, for example, 8:00 a.m., 9:00 a.m., etc. The plurality of discrete scheduled times refer to the times set in accordance with the predetermined rule, for example, the scheduled times every two hours, the times every two hours from 6:00, 8:00, . . . to 24:00. Through the solution described above, it is possible to reduce the amounts of data acquisition and transmitting.

In this embodiment, the plurality of air quality detection units 10 corresponding to the plurality of vehicles 1 (see FIG. 1) use the same schedule. As previously described, when the air quality map is drawn on the side of server 200 (see FIG. 1), it is required that the data is aligned or substantially aligned in time. Through the solution described above, this embodiment can easily realize the alignment in time of the data between the different vehicles, which facilitates the map drawing on the side of server 200 (see FIG. 1).

With reference to FIG. 1, in this embodiment, the system further comprises a server 200 already described above. As previously described, the server 200 may be configured to form an air quality map according to the air quality data from the plurality of vehicles 1.

In one embodiment, the air quality map may be formed by correlating the position data with latitude and longitude coordinates in the map based on the position data and the air quality data at the current position of the vehicle 1 and marking the air quality data corresponding to the position data. By calculating, after further weighted averaging, the overall air quality data in the area and making a contour map of the overall air quality data (which may be the overall PM2.5 value), the general air pollution indexes of the latitudes and longitudes throughout the country can be known as needed. Through the solution described above, this embodiment greatly facilitates the users and provide more accurate guidance for their travel and life.

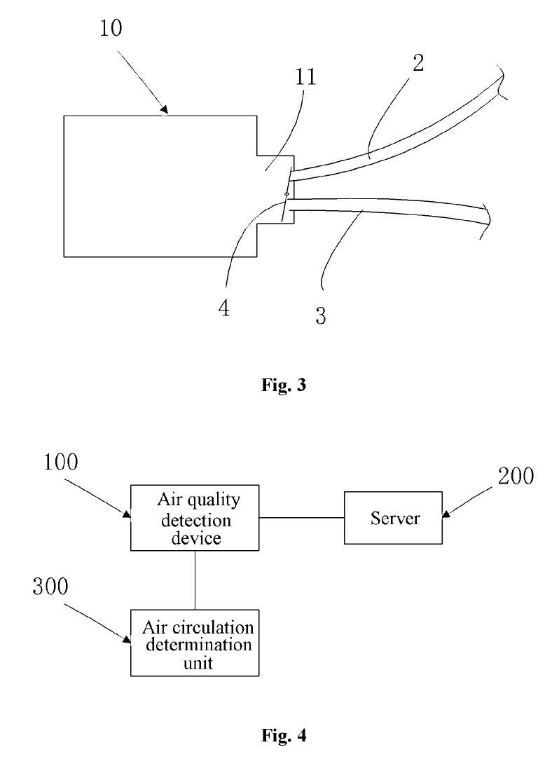

FIG. 3 is a schematic assembly diagram of the air quality detection unit 10 shown in FIG. 2 with an outer air inlet pipe 2 and an inner air inlet pipe 3 to represent the arrangement condition of the air quality detection unit 10 at the vehicle 1 (see FIG. 1). As shown in FIG. 3, in this embodiment, the air quality detection unit 10 has an inlet port 11. The inlet port 11 is in communication with the outer air inlet pipe 2 and the inner air inlet pipe 3 separately through a one-way valve 4, an air inlet of the outer air inlet pipe 2 is provided outside the vehicle 1 (see FIG. 1), and an air inlet of the inner air inlet pipe 3 is provided inside the vehicle 1 (see FIG. 1), and by selecting the opening direction of the one-way valve 4, the inlet port 11 is in communication with one of the outer air inlet pipe 2 and the inner air inlet pipe 3 so as to correspondingly acquire the air quality data outside the vehicle 1 (see FIG. 1) or acquire the in-vehicle air quality data inside the vehicle 1 (see FIG. 1). In the specific implementation, the control of the one-way valve 4 can be switched manually by the driver or automatically by the computer periodically, so that it can be automatically displayed on the multimedia screen in the vehicle 1 (see FIG. 1).

With reference to FIG. 3, in this embodiment, the air inlet of the outer air inlet pipe 2 is provided at an intake grille of the vehicle 1 (see FIG. 1). In other embodiments, the air inlet of the outer air inlet pipe 2 may otherwise be provided in a transition region between a windshield and an engine hood of the vehicle 1 (see FIG. 1), the transition region between the windshield and the engine hood of the vehicle 1 (see FIG. 1) has a certain waterproof and dustproof area, thereby avoiding the possibility of secondary contamination of the air quality detection unit 10, keeping the air inlet of the outer air inlet pipe 2 clean, and helping to ensure the accuracy of the air quality data.

The air quality detection unit 10 can be obtained by modifying the existing vehicle-mounted air quality detector, so that the air quality detection unit 10 can acquire both the air quality data outside the vehicle 1, and also acquire the air quality data inside the vehicle 1, thereby having the advantages of simple operation and convenient use. In other embodiments, the air quality detection unit 10 may also be dedicated to detecting the air quality data outside the vehicle 1. The air quality detection unit 10 may communicate with the data transmission unit 20 in a wireless or wired manner.

The previously described air quality detection device 100 can be completed as a built-in device at the time of vehicle design and manufacture, or as an additional device installed on an existing vehicle, thereby improving the flexibility of the solution implementation.

FIG. 4 is another schematic logic control diagram of the system shown in FIG. 1. As shown in FIG. 4, the system may further comprise an air circulation determination unit 300 for determining whether or not the door or the window of the vehicle 1 (see FIG. 1) has been in an open state for a predetermined time. The air quality detection unit 10 (see FIG. 2) may be installed inside the vehicle 1 (see FIG. 1) and acquire the in-vehicle air quality data inside the vehicle 1 (see FIG. 1). The data transmission unit 20 (see FIG. 2) can, if the result of the determination of the air circulation determination unit 300 is positive, transmit the in-vehicle air quality data as the air quality data outside the vehicle 1 (see FIG. 1) to the server 200.

In this embodiment, the air quality detection unit 10 (see FIG. 2) can mainly detect the air quality inside the vehicle 1 (see FIG. 1) and visually display the change of air quality inside the vehicle 1 (see FIG. 1) to the user. The air quality detection unit 10 (see FIG. 2) does not directly detect the air quality outside the vehicle 1. However, the air circulation determination unit 300 makes a determination for the state of the door or the window by receiving a signal of the door switch or the window switch, if the signal displays that the door or the window is opened for a period of time T≥A seconds, the in-vehicle air quality data can be used as the air quality data outside the vehicle 1 (see FIG. 1).

In this way, the air quality data outside the vehicle can be indirectly obtained by means of the air quality detection unit built in a vehicle in the prior art for detecting the air quality data inside the vehicle, so that this solution does not require modification of the existing air quality detection unit, thus eliminating the need for additional costs.

The air quality data in the embodiments described above may be or include PM2.5 data. In this embodiment, the air quality data mainly includes PM2.5 data, but may also include other air quality data, such as nitrogen oxide data and sulfide data, thereby acquiring the concentration of the pollutant outside the vehicle 1 and further feeding back the concentration value of nitrogen oxides, sulfides and other pollutants except for PM2.5 to the users.

In addition, the principle of the air quality detection system of the present invention is not only applicable to the Internet of Vehicles, but also to the detection of the air quality of the user's household purifier and the air quality of the user's mobile phone side, and by installing air quality monitoring devices on the user's household purifier and the user's mobile phone to detect the air quality data of the user's home environment and the user's activity environment, so that the user can clearly know and choose which environment to work, live and travel, thereby improving the user experience.

In addition, it is possible to obtain the difference between the air quality map and the government release information by comparing the air quality map made from the air quality data of the system of the present invention with the PM2.5 concentration released from the national government, so as to correct the air quality map or display the accuracy of the air quality map to the user.

Up to this, a person skilled in the art should recognize that although a plurality of exemplary embodiments of the present invention have been shown and described in detail herein, numerous other variations or modifications meeting the principle of the present invention can be directly determined or derived according to the contents disclosed in the present invention. Therefore, the scope of the present invention should be construed and considered as covering all of such other variations or modifications.

1. An internet of vehicles-based air quality detecting system, characterized by comprising an air quality detection device arranged in each of a plurality of vehicles, the air quality detection device comprising:
an air quality detection unit for acquiring air quality data outside the vehicle at a current position where the corresponding vehicle is located; and
a data transmission unit for transmitting the air quality data to a remote server.

2. The system according to claim 1, characterized in that the air quality detection device further comprises a position acquisition unit for acquiring position data of the current position where the corresponding vehicle is located,
wherein the data transmission unit is configured to transmit the position data of the current position to the server together with the air quality data of the current position.

3. The system according to claim 1, characterized in that the air quality detection device further comprises a time acquisition unit for acquiring time data of a moment when the corresponding vehicle is located at the current position,
wherein the data transmission unit is configured to transmit the time data of the current position to the server together with the air quality data of the current position.

4. The system according to claim 1, characterized in that the air quality detection unit is configured to acquire the air quality data corresponding to a schedule, which comprises one or more discrete scheduled time; and
the data transmission unit is configured to transmit only the air quality data corresponding to the schedule to the server.

5. The system according to claim 4, characterized in that the plurality of air quality detection units corresponding to the plurality of vehicles use the same schedule.

6. The system according to claim 1, characterized by further comprising the server, which is configured to form an air quality map according to the air quality data from the plurality of vehicles.

7. The system according to claim 1, characterized in that the air quality detection unit has an inlet port, which is in communication with an outer air inlet pipe and an inner air inlet pipe separately through a one-way valve, an air inlet of the outer air inlet pipe is provided outside the vehicle, and an air inlet of the inner air inlet pipe is provided inside the vehicle, and by selecting the opening direction of the one-way valve, the inlet port is in communication with one of the outer air inlet pipe and the inner air inlet pipe so as to correspondingly acquire the air quality data outside the vehicle or acquire the in-vehicle air quality data inside the vehicle.

8. The system according to claim 7, characterized in that the air inlet of the outer air inlet pipe is provided at an intake grille of the vehicle or in a transition region between a windshield and an engine hood of the vehicle.

9. The system according to claim 1, characterized by further comprising:
an air circulation determination unit for determining whether or not a door or a window of the vehicle has been in an open state for a predetermined time,
wherein the air quality detection unit is installed inside the vehicle and acquires in-vehicle air quality data inside the vehicle; and the data transmission unit is configured to, if the result of the determination of the air circulation determination unit is positive, transmit the in-vehicle air quality data as the air quality data outside the vehicle to the server.

10. The system according to claim 1, characterized in that the air quality data is or comprises PM2.5 data.
